(19) United States
(12) Patent Application Publication
(10) Pub. No.: US 2016/0166268 A1
(43) Pub. Date: Jun. 16, 2016

(54) DEVICE FOR REMOVING A FOREIGN OBJECT FROM A BODILY ORIFICE

(71) Applicant: Justin Charles TOPPINS, Hendersonville, TN (US)

(72) Inventor: Justin Charles TOPPINS, Hendersonville, TN (US)

(21) Appl. No.: 14/954,006

(22) Filed: Nov. 30, 2015

Related U.S. Application Data
(60) Provisional application No. 62/090,861, filed on Dec. 11, 2014.

(51) Int. Cl. A61B 17/221; A61B 17/50

(52) U.S. Cl. CPC A61B 17/221; A61B 17/50; A61F 11/006

(57) ABSTRACT

A device for removing a foreign object from a bodily orifice has a first tubular body, a second tubular body connected to the first tubular body, and a line extending through an interior of the first tubular body and through an interior passageway of the second tubular body. The line defines a loop extending outwardly of the far end of the second tubular body. The line has at least one end extending outwardly of a proximal end of the first tubular body. A grip member is affixed to the end of the line. The grip member is movable relative to the first tubular body and the second tubular body so as to increase or decrease a diameter of the loop of the line. The grip member is rotatably movable relative to the first tubular body and the second tubular body so as to rotated the loop of the line.

### CROSS-REFERENCE TO RELATED APPLICATIONS

The present application claims priority from U.S. Provisional Patent Application Ser. No. 62/090,861, filed on Dec. 11, 2014, and entitled “FB Loop”.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

1. Field of the Invention

The present invention relates to devices that remove foreign objects from bodily orifices, such as noses and ears. More particularly, the present invention the relates to devices for removing foreign objects that include an adjustable loop at an end thereof such that the adjustable loop can secured around the foreign object.

2. Description of Related Art Including Information Disclosed Under 37 CFR 1.97 and 37 CFR 1.98.

Human nature leads children, and sometimes adults, to put objects where they do not belong. All too frequently, these places are part of the human body. Nasal cavities, ear canals and throats of the most common areas into which objects are placed haphazardly or accidentally lodged, causing discomfort, injury and occasionally, serious injury. Physicians frequently are visited by children having potentially dangerous foreign objects lodged in these or other places.

Forceps are the most common device for removing such foreign objects from the passageway being blocked. However, forceps can be damaging to the sensitive tissues which often make up these passageways and, thus, further damage may result during the attempted removal of the object. This method is often lengthy and traumatic for a child, and it may be unsuccessful, resulting in surgery.

An ear curette instrument is commonly used to remove cerumen and foreign bodies from ear canals. Most such curettes have a long, stiff, relatively thick straight shaft of metal or plastic to the front of which is fixedly connected in a set position a relatively large wire loop or spoon of fixed size tilted at an angle to allow it to be positioned behind the cerumen or foreign material in the ear canal. The cerumen or foreign object is extracted by pulling it out of the ear canal as the instrument is withdrawn.

Certain problems are encountered with such instruments. The fixed angle and large cross-sectional diameter of the instrument tip hinders the passage of the tip through the distal orifice with large otoscope specula. Such task is impossible when ears speculums of appropriate small size are used on infants and small children. Moreover, once the instrument tip is advanced beyond the otoscope speculum, it is difficult to avoid pushing the cerumen or foreign object deeper into the ear canal.

Proper alignment of the instrument tip by advancing it downward is very difficult due to the size of the otoscope head. In addition, the instrument is difficult to stabilize if the patient moves, thereby increasing the risk of abrasion or laceration of the ear canal or perforation of the eardrum. Such an instrument largely obstructs a clear view of the eardrum, walls of the ear canal and the cerumen or foreign object because of the large diameter of the instrument shaft, the fixed angle of the tip, the required angling of the tip and because the practitioner's hand blocks the view.

Current foreign body removal devices, such as the curette, are firm non-pliable devices. They work by forcing the user to manually push into the ear in an attempt to get behind the foreign body and then apply pressure downward to remove the foreign body. This is problematic on several levels. First, there is a risk of pushing the object further into the orifice. In the case of the ear, this can result in trauma, such as tympanic membrane rupture. There is also a great risk for discomfort if the object is sharp or jagged. In cases like this, the patient will likely be unable to tolerate the procedure. This can result in a failed procedure. As a result, further follow-up work is required and additional costs. The curettes currently available do not provide a comfortable and safe way to firmly grasp the foreign body.

In the past, various patents have issued relating to devices for the removal of objects from body cavities, such as noses and ears. For example, U.S. Pat. No. 4,572,180, issued on Feb. 25, 1986 to R. P. Denadayalu, describes a lighted ear canal curette instrument which is adapted for use in removing an undesirable substance from either an ear canal or a nasal passage. The lighted curette comprises a lighting member to which is attached a handle from which extends a stem. The stem has an engagement member adjacent the end thereof for engaging and removing an undesirable substance from the nasal passage or ear canal.

U.S. Pat. No. 5,133,721, issued on Jul. 28, 1992 to E. D. Angulo, teaches a device for removing foreign objects from anatomic organs, such as the ear canal or throat. This device has a housing shaped like a flashlight, an electrical power source, and a tip extending from the housing. The tip has at least one wire loop made from a shape-memory-effect alloy switchably connected the electrical power source such that when the electric current flows through the wire loop, the wire loop heats up and returns to a previously programmed shape, such as a curette or tweezers, so as to facilitate removal of the foreign object.

U.S. Pat. No. 5,171,233, issued on Dec. 15, 1992 to Amplatz et al., provides a snare-type probe. The snare is utilized in intravascular use and has an elongate proximal member and a loop-shaped distal segment oriented at an angle to the adjacent portion of the proximal member. The snarei s in the form of a super elastic-shape-memory alloy. This permits the distal segment to be collapsed for passage through a catheter and automatically open into its original, unrestrained configuration upon emerging from the distal tip of the catheter.

U.S. Pat. No. 5,390,663, issued on Feb. 21, 1995 to N. E. Schafer, discloses a canal obstruction remover in the form of an ear speculum that incorporates one or more protrusions that move independently of the body of the speculum. The protrusions can be bent back toward the distal end of the speculum. Bending is accomplished by pulling a filament attached to the end of the protrusion. The filament may be pulled manually or with the aid of a trigger or a rotating knob.

U.S. Pat. No. 5,454,817, issued on Oct. 3, 1995 to D. L. Katz, teaches a foreign body extracting device that includes an elongated flexible tubular rod having a compressible bulb attached to its proximal end. The bulb is in fluid communication with the rod in order to pump air through the rod. An inflatable balloon is attached to a distal end of the tubular rod and is in fluid communication with the rod. The balloon is positioned and connected with the rod so as to receive the air from the bulb and, as a result, become inflated. The extractor also includes a handgrip which is used for holding the extractor, compressing the bulb and maintaining compression with one hand.

U.S. Pat. No. 6,074,405, issued on Jun. 13, 2000 to C. S. Koch, provides a medical instrument from removing the lumen obstructions. This instrument includes an elongated handle with an elongated hollow tube connected to the front end thereof and an elongated flexible wire extending from the front end of the tube. The front end of the wire is curved to form a scoop for removing obstructions from a lumen. The handle has a central cavity that communicates with the cavity in the tube and through both of which the wire extends. The wire is connected to a spring which extends out of the handle. When the wire is manually depressed, the front end of the wire extends out of the tube and curves into the desired scoop configuration.

U.S. Pat. No. 6,017,353, issued on Jan. 25, 2000 to R Rankings, shows an instrument for retrieving foreign bodies from an ear or a nose canal. This instrument has a hollow tubular body with a longitudinal passageway therein and a rod with a plurality of flexible members attached at one end. In the closed position, the rod and flexible members are received within the passageway and the flexible members are collapsed against each other. In the open position, the flexible members extend beyond the end of the tubular body and expand apart. The ends of the flexible members have an angled portion for grasping and retrieving the foreign body. The instrument is placed in the ear or nose canal in the closed position. The tubular body is slid toward the rod end of the instrument causing the flexible members to expand against the ear or nose canal wall. The entire instrument is pushed into the ear or nose canal until the angled portions go beyond the stuck foreign body. The instrument and foreign body are then pulled from the ear or nose.

U.S. Pat. No. 6,168,604, issued on Jan. 2, 2001 to G. G. Cano, discloses a guide wire device for removing solid objects from body canals. A vascular filter captures and removes emboli and includes a sack having a mouth in a closed bottom opposite the mouth. A guide wire is received through the mouth of the sack and projects through the closed bottom of the sack. The closed bottom of the sack is connected to the projection of the guide wire therein. A collapsible frame is connected between the guide wire and the mouth of the sack. The collapsible frame biases the mouth of the sack open around the guide wire. A tube slidably receives the guide wire coaxially therein. The collapsible frame is movable via the guide wire between outside the tube where the mouth of the sack is biased open by the collapsible frame when the tube or the mouth of the sack is closed, and vice versa.

U.S. Pat. No. 6,699,178, issued on Mar. 2, 2004 to Y. Koda, discloses an endoscopic auditory canal cleaning apparatus that includes an ear-pick main body which is equipped with a scraping part at its distal end and is formed to guide light to the distal end. A light source generates the light. The light passes through a hollow opening provided in an opening part. The holding part holds the fiber scope so as to be able to rotate freely adjacent to a scoop.

U.S. Pat. No. 8,728,107, issued on May 2, 2014 to Huttner et al., teaches a method of removing cerumen or a foreign body from an ear canal. This method includes providing an ear curette having a curette connected to first and second actuator arms, introducing the curette tip into the ear canal, actuating the ear curette by forcing the first and second actuator arms in opposite directions as to cause the movement of the curette tip about the first and second living hinges, and withdrawing the curette tip and at least a portion of the cerumen for the foreign body from the ear canal. The curette tip engages at least a portion of the cerumen or the foreign body in the ear canal.

It is an object of the present invention to provide a device that removes foreign bodies from bodily orifices, such as nasal passages and ear canals.

It is another object of the present invention to provide a device that has the ability to go behind or around the foreign body.

It is another object of the present invention to provide a device that avoids pushing the foreign body deeper into the orifice.

It is another object of the present invention to provide a device that avoids harming the person during the procedure.

It is another object of the present invention to provide a device for removing a foreign object which is gentle to the patient.

It is another object of the present invention to provide a device effectively removes cerumen impactions.

It is still a further object of the present invention to provide a device that avoids the need for flushing activities.

It is still a further object of the present invention to provide a device for removing foreign bodies from a nasal cavity or ear canal which avoids trauma to the patient.

These and other objects and advantages of the present invention will become apparent from a reading of the attached specification and appended claims.

### BRIEF SUMMARY OF THE INVENTION

The present invention is a device for removing a foreign object from a bodily orifice. The device comprises a first tubular body having a proximal end and a distal end, a second tubular body connected to the first tubular body and having a near end and a far end, and a line extending through an interior of the first tubular body and through an interior passageway of the second tubular body. The line defines a loop extending outwardly of the far end of the second tubular body. The line has at least one end extending outwardly of the proximal end of the first tubular body.

A grip member is affixed to this one end of the line. The grip member is movable relative to the first tubular body and the second tubular body so as to increase or decrease a diameter of the loop of the line. The grip member is positioned outwardly of the proximal end of the first tubular body. Preferably, the grip member is also rotatably movable relative to the first and second tubular bodies so as to rotate the loop of the line.

The far end of the second tubular body extends outwardly beyond the distal end of the first tubular body. The second tubular body has a pliability that is greater than a pliability of the first tubular body. The line has a pliability that is greater than a pliability of the second tubular body.

The first tubular body comprises an outer tube and an inner tube extending outwardly beyond an end of the outer tube. The second tubular body extends outwardly of an end of the inner tube opposite the outer tube. The inner tube has a pliability that is greater than a pliability the outer tube. A rigid tube is positioned within the outer tube. This rigid tube extends from the proximal end of the first tubular body toward the inner tube. The outer tube is formed of a clear polymeric material.

In the present invention, the line has a first end and a second end extending outwardly of the proximal end of the first tubular body. The grip member is affixed to the first and second ends of the line. The second tubular body has an outer diameter that is less than an outer diameter of the first tubular body. Each of the first tubular body and the second tubular body are of an elongated longitudinal shape. The longitudinal axis of the first tubular body is axially aligned with the longitudinal axis of the second tubular body.

This foregoing Section is intended to describe, with particularity, the preferred embodiments of the present invention. It is understood that modifications to these preferred embodiments can be made within the scope of the present claims. As such, this Section should not to be construed, in any way, as limiting of the broad scope of the present invention. The present invention should only be limited by the following claims and their legal equivalents.

### BRIEF DESCRIPTION OF THE SEVERAL VIEWS OF THE DRAWINGS

FIG. 1 is a cross-sectional view showing the device for removing foreign objects from a bodily orifice in accordance with the preferred embodiment of the present invention.

FIG. 2 is an exploded view showing the device for removing foreign objects from a bodily orifice in accordance with the preferred embodiment of the present invention.

FIG. 3 is a cross-sectional view showing the enlarging of the diameter of the loop of the line associated with the device for removing foreign objects from bodily orifices of the preferred embodiment of the present invention.

FIG. 4 is a cross-sectional view showing the rotation of the loop of the line associated with the device for removing foreign objects from bodily orifices of the preferred embodiment of the present invention.

### DETAILED DESCRIPTION OF THE INVENTION

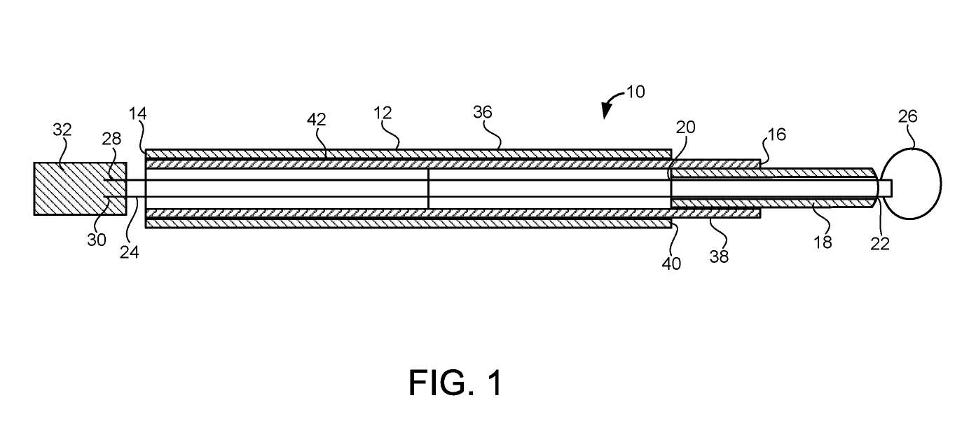

Referring to FIG. 1, there is shown the device 10 for removing foreign objects from bodily orifice. In particular, the device 10 is intended to remove objects from a nasal passage or from an ear canal. However, within the concept of the present invention, the device can be used in association with other bodily orifices, including human and animal.

FIG. 1 shows the device 10 as having a first tubular body 12 having a proximal end 14 and a distal end 16. As can be seen, the first tubular body 12 has an interior. A second tubular body 18 is connected to the first tubular body 12. The second tubular body 18 has a near end 20 and a far end 22. The second tubular body 18 also has an interior passageway. A line 24 extends through the interior of the first tubular body 12 and the interior passageway of the second tubular body 18. The line 24 defines a loop 26 extending outwardly of the far end 22 of the second tubular body 18. The line 24 includes a first end 28 and a second end 30 that extend outwardly beyond the proximal end 14 of the first tubular body 12. A grip member 32 is affixed to the ends 28 and 30 of the line 24.

The grip member 32 is movable relative to the first tubular body 12 and the second tubular body 18 so as to increase or decrease a diameter of the loop 26 of the line 24. The grip member 32 is positioned outwardly of the proximal end 14 of the first tubular body 12. Typically, the grip member 32 would be in the nature of a plastic piece that can be molded onto the ends 28 and 30 of the line 24. The grip member 32 is intended to be held with one hand while the first tubular body 12 is held with another hand. As such, the first tubular body 12 can be interpreted as a “handle” used in the present invention.

The second tubular body 18 has the far end 22 that extends outwardly beyond the distal and 16 of the first tubular body. The second tubular body 18 will have a pliability that is greater than a pliability of the first tubular body 12. Additionally, the line 24 will also have a pliability that is greater than the pliability of the second tubular body 18. In other words, the second tubular body 18 can be formed of a soft polymeric material. The line 24 can be a plastic line, such as nylon, or plastic wire. The pliabilities of these elements are very significant in the present invention. Ultimately, since the loop 26 extends furthest into the orifice, it should be as soft as possible so as to avoid damaging those distant tissues, such as an eardrum. Additionally, since the second tubular body 18 also penetrates deeply into the orifice, it should be constructed of a very soft pliable material so as to avoid damaging surrounding tissues. Ultimately, the pliabilities of the line 28 and the second tubular body 18 create a synergistic effect which allows the device 10 to carry out proper performance while creating a “stepped” nature of pliability so as to avoid damage and trauma the surrounding tissues and to avoid perforation.

The first tubular body 12 has an outer tube 36 and an inner tube 38. The inner tube extends outwardly of an end 40 of the outer tube 12. The second tubular body 18 extends outwardly beyond the end 16 of the inner tube 38. The inner tube 38 also has a pliability that is greater than a pliability the outer tube 36. Once again, this enhances the “stepped” nature of pliability in the relationship between the inner tube 38, the second tubular body 18 and the line 24. Since the inner tube 38 may penetrate into the outer surfaces of the orifice, it should be suitably pliable to avoid damaging such tissues. Since such tissues are adjacent the outer surface of the orifice, it is less likely that these would be damaged by a more rigid tubular member, such as the inner tube 38. The rigid outer tube 36 will ultimately reside outside of the orifice and, as such, would have no possible damaging effect to tissues.

For the purposes of guidability and control, a rigid tube 42 is positioned within the outer tube 36. The rigid tube 42 is illustrated as extending from the proximal end 14 of the first tubular body 12 toward the end of the inner tube 38. The use of this rigid tube 42 enhances the structural integrity of the outer tube 36 and the structural integrity of the first tubular body 12 in those areas where the device 10 is handled. As such, this enhances the ability of the first tubular body 12 the function as a handle. Ultimately, the line 24 is illustrated as extending through the interior of the rigid tube 42. Rigid tube 42 can be made of a metal material, such as steel or aluminum. Other materials can be employed, as required. The outer tube 36 can be formed of a clear polymeric material.

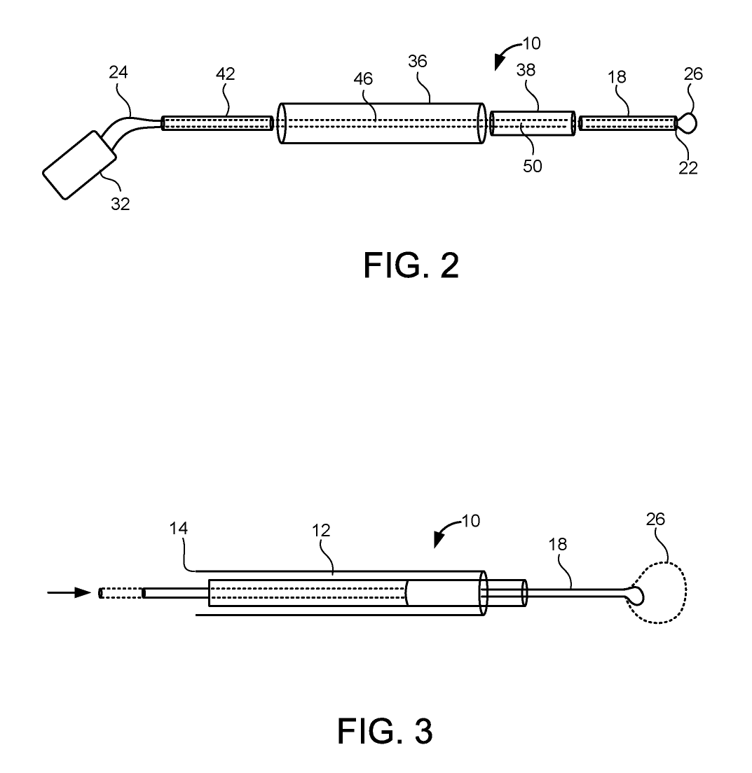

FIG. 2 shows an exploded view of the device 10 of the present invention. In FIG. 2, it can be seen that the outer tube 36 has an interior 46 which serves to accommodate the rigid tube 42 and an end of the inner tube 38. The inner tube 38 has an interior passageway 50. The second tubular body 18 also has an interior passageway. The loop 26 extends outwardly of the distal end 22 of the second tubular body 18. The line 24 is illustrated as affixed to the grip member 32 and extends continuously through the interior of the rigid tube 42, through the interior 46 of the outer tube 36 and through the interior air passageway 50 of the inner tube 38. Ultimately, line 28 will extend through the interior passageway of the second tubular body 18 so as to emerge outwardly of the distal end 22 in the form of a loop 26. The loop 26 is of a generally small diameter. The small diameter is particularly configured to grip the foreign object and/or cerumen. This is achieved by pulling on the gripping member 32 so as to retract the line toward the proximal end 14 of the first tubular body 12.

FIG. 3 shows the device 10 in which the gripping member 32 is pushed toward the proximal end 14 of the first tubular body 12. This urges the line through the interiors of the first tubular body 12 and the second tubular body 18 so that the loop 26 is of an increased diameter. This increased diameter loop 26 is configured so as to “lasso” the foreign object and/or cerumen. Once this lasso has been placed over the foreign object, the gripping member 32 is pulled away from the proximal end 14 of the first tubular body 12 so as to reduce the diameter of the loop 26 so that the object is effectively lassoed. The entire device 10 can then be removed from the bodily orifice.

The loop (or curette) expands once in place behind a lodged foreign body. This ensures that the foreign object can be removed from this lodged position.

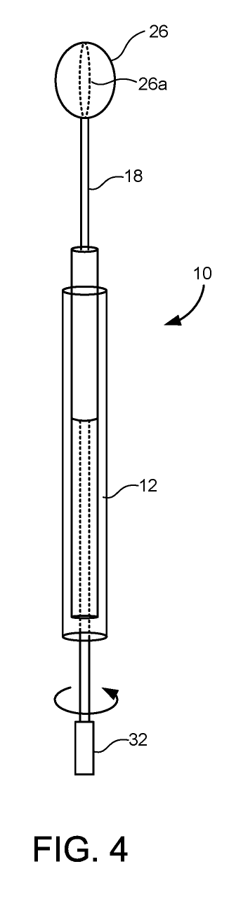

Referring to FIG. 4, it can be seen how the gripping member 32 can be rotated relative to the first tubular body 12. This rotation of the gripping member 32 causes the loop 26 to rotate to a position shown by reference numeral 26a. Rotation of the loop 26 allows for greater maneuverability and mobility of the device in closed spaces as compared to the prior art. This rotation makes it easier for the user to navigate around and subsequently capture the foreign object and/or cerumen.

The device 10 provides distinctive advantages over current curettes used for foreign body removal in orifices of the body, such as the nose and/or ears. The loop 26 (or curette) is thinner at the tip than the typical models. The loop's ability to expand and rotate is controlled with a small plastic grip at the end of the device. This allows the user to go behind or around the foreign body. This technique ensures that the object can be removed and makes it less likely that it will be pushed further into the orifice. The tip of the second tubular body 18 is firm, but pliable. This offers another advantage over the prior art in that it limits the risk of harming the patient during the process of foreign body removal. Unlike the rigid devices the prior art, the flexibility of the present invention avoids to the need to generate more pressure in order to get the curette in a posterior position on the foreign body. As such, this minimizes the risk for injury to the patient, such as eardrum perforation. The present invention enhances the ability to gently remove the foreign body from the orifice.

The device 10 of the present invention can also be used for cerumen impactions. The curette tip is thin and can easily pass behind the impaction so as to allow for breakdown and removal posterior to the impaction. This further limits the risk of perforation of the eardrum and avoids the discomfort association with flushing techniques.

The foregoing disclosure and description of the invention is illustrative and explanatory thereof. Various changes in the details of the illustrated construction can be made within the scope of the appended claims without departing from the true spirit of the invention. The present invention should only be limited by the following claims and their legal equivalents.

I claim:

1. A device for removing a foreign object from a bodily orifice, the device comprising:
a first tubular body having a proximal end and a distal end and an interior;
a second tubular body connected to said first tubular body, said second tubular body having a near and at a far end and an interior passageway; and
a line extending through said interior of said first tubular body and said interior passageway of said second tubular body, said line defining a loop extending outwardly of said far end of said second tubular body, said line having at least one end extending outwardly of said proximal end of said first tubular body.

2. The device of claim 1, further comprising:
a grip member affixed to said at least one end of said line.

3. The device of claim 2, said grip member movable relative to said first tubular body and said second tubular body so as to increase or decrease a diameter of said loop of said line.

4. The device of claim 2, said grip member positioned outwardly of said proximal end of said first tubular body.

5. The device of claim 1, said far end of said second tubular body extending outwardly beyond said distal end of said first tubular body.

6. The device of claim 1, said second tubular body having a pliability that is greater than a pliability of said first tubular body.

7. The device of claim 6, said line having a pliability that is greater than a pliability of said second tubular body.

8. The device of claim 1, said first tubular body comprising:
an outer tube; and
an inner tube extending outwardly beyond an end of said outer tube, said second tubular body extending outwardly of an end of said inner tube opposite said outer tube.

9. The device of claim 8, said inner tube having a pliability that is greater than a pliability of said outer tube.

10. The device of claim 8, further comprising:
a rigid tube positioned within said outer tube, said rigid tube extending from said proximal end of said first tubular body toward said inner tube.

11. The device of claim 8, said outer tube being formed of a clear polymeric material.

12. The device of claim 2, said grip member rotatably movable relative to said first tubular body and said second tubular body so as to rotate said loop of said line.

13. A device for removing a foreign object from a bodily orifice, the device comprising:
a handle having a proximal end and a distal end and an interior;
a tubular member extending outwardly of said distal end of said handle, said tubular member having a near end and a far end;
a line defining a loop outwardly of said far end of said tubular member, said line extending through an interior passageway of said tubular member and through said interior of said handle, said line having at least one end extending outwardly of said proximal end of said handle; and
a grip member affixed to said at least one end of said line, said grip member movable toward said handle so as to increase a diameter of said loop, said grip member movable away from said handle so as to decrease the diameter of said loop.

14. The device of claim 13, said tubular member having a pliability that is greater than a pliability of said handle.

15. The device of claim 13, said handle comprising:
an outer tube; and
an inner tube extending outwardly beyond an end of said outer tube, said tubular member extending outwardly of an end of said inner tube opposite said outer tube.

16. The device of claim 15, said inner tube having a pliability that is greater than a pliability of said outer tube.

17. The device of claim 15, further comprising:
a rigid tube positioned within said outer tube, said rigid tube extending from said proximal end of said handle toward said inner tube.

18. The device of claim 15, said grip member rotatably movable relative to said handle so as to rotate said loop of said line.

19. The device of claim 13, said tubular member having an outer diameter less than an outer diameter of said handle.

20. The device of claim 13, each of said handle and said tubular member being of an elongated longitudinal shape, said tubular member having a longitudinal axis axially aligned with a longitudinal axis of said handle.
